(19) United States
(12) Patent Application Publication
(10) Pub. No.: US 2012/0325028 A1
(43) Pub. Date: Dec. 27, 2012

(54) SYNTHETIC RESIN MATERIAL TEST METHOD AND SYNTHETIC RESIN MATERIAL TEST APPARATUS

(76) Inventors: Akira Tanaka, Tokyo (JP); Kaoru Yamaoka, Tokyo (JP)

(21) Appl. No.: 13/443,564

(22) Filed: Apr. 10, 2012

(30) Foreign Application Priority Data
Jun. 24, 2011 (JP) 2011-140560

(51) Int. Cl. G01N 33/00

(52) U.S. Cl. 73/866

(57) ABSTRACT

According to one embodiment, a synthetic resin material test method includes: forming a first plane on a sample made of first synthetic resin material by cutting or grinding the sample; obtaining measurement values at a plurality of positions on the first plane by pressing the first plane at the positions; and outputting the measurement values or physical quantities calculated based on the measurement values, in association with the respective positions.

### CROSS-REFERENCE TO RELATED APPLICATIONS

This application is based upon and claims the benefit of priority from Japanese Patent Application No. 2011-140560, filed on Jun. 24, 2011, the entire contents of which are incorporated herein by reference.

### FIELD

Embodiments described herein relate generally to a synthetic resin material test method and a synthetic resin material test apparatus.

### BACKGROUND

There have been known test methods in which a sample made of synthetic resin material is tested on the basis of reflected light or transmitted light from the sample irradiated with light.

In the synthetic resin material test methods, there is a demand to obtain test results with higher accuracy.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention.

FIG. 1 is an exemplary schematic plan view of a test apparatus that implements a synthetic resin material test method according to an embodiment;

FIG. 2 is an exemplary perspective view of a base and a sample used in the synthetic resin material test method in the embodiment;

FIG. 3 is an exemplary flowchart of a procedure of the synthetic resin material test method in the embodiment;

FIG. 4 is an exemplary cross-sectional view of the base illustrated in FIG. 2, in the embodiment;

FIG. 5 is an exemplary cross-sectional view of the base illustrated in FIG. 2 when the sample is set in the base, in the embodiment;

FIG. 6 is an exemplary cross-sectional view of the base and the sample illustrated in FIG. 5 when synthetic resin material is filled and solidified in the base, in the embodiment;

FIG. 7 is an exemplary cross-sectional view illustrating a position to be processed of the base and the sample illustrated in FIG. 6, in the embodiment;

FIG. 8 is an exemplary cross-sectional view of the sample with a plane formed at the processing position illustrated in FIG. 7, in the embodiment;

FIG. 9 is an exemplary schematic diagram of a part of a measurement device used for the sample to which the plane illustrated in FIG. 8 is formed, in the embodiment;

FIG. 10 is an exemplary schematic plan view of measurement positions of the sample in the synthetic resin material test method in the embodiment;

FIG. 11 is an exemplary plan view of a movement device that moves the measurement positions of the sample in the synthetic resin material test method in the embodiment;

FIG. 12 is an exemplary diagram of a presser (side view) and the sample (cross-sectional view) in the synthetic resin material test method before the presser presses the sample, in the embodiment;

FIG. 13 is an exemplary diagram of the presser (side view) and the sample (cross-sectional view) in the synthetic resin material test method when the presser presses the sample, in the embodiment;

FIG. 14 is an exemplary diagram of the presser (side view) and the sample (cross-sectional view) in the synthetic resin material test method when the presser is separated from the sample after the presser presses the sample, in the embodiment;

FIG. 15 is an exemplary diagram illustrating an output, in which physical quantities, which are calculated based on a measurement result obtained in the synthetic resin material test method, are depicted in association with the measurement positions, in the embodiment;

FIG. 16 is an exemplary schematic perspective view of weld lines and cracks generated on the sample corresponding to the output in FIG. 15, in the embodiment; and

FIG. 17 is an exemplary cross-sectional view of the base with the sample illustrated in FIG. 8, with illustration of processing positions (positions where planes are formed), in the embodiment.

### DETAILED DESCRIPTION

In general, according to one embodiment, a synthetic resin material test method comprises: forming a first plane on a sample made of first synthetic resin material by cutting or grinding the sample; obtaining measurement values at a plurality of positions on the first plane by pressing the first plane at the positions; and outputting the measurement values or physical quantities calculated based on the measurement values, in association with the respective positions.

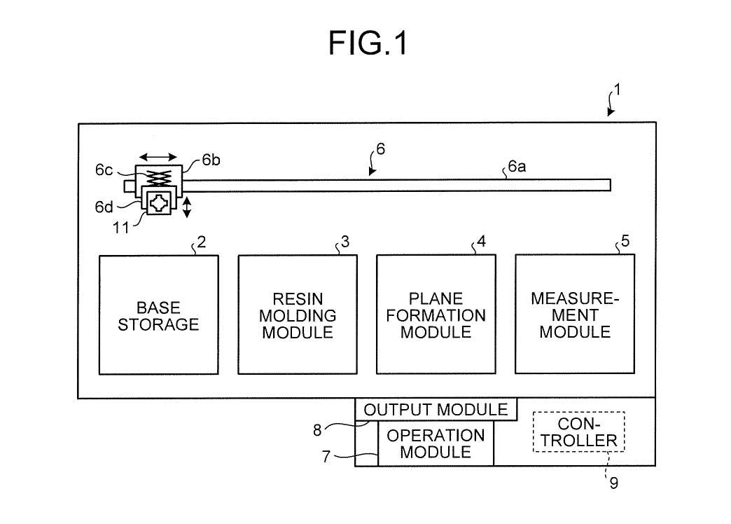

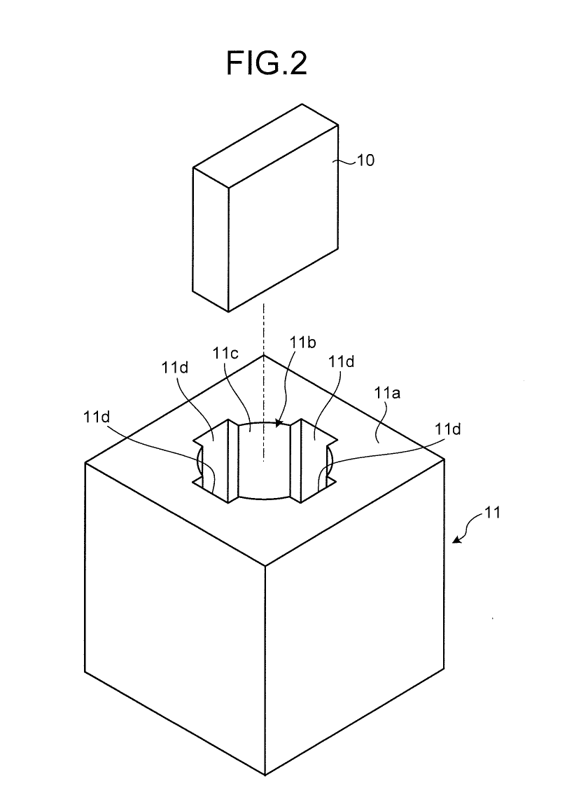

As illustrated in FIG. 1, a test apparatus 1 comprises abase storage 2, a resin molding module 3, a plane formation module 4, a measurement module 5, a conveyor 6, an operation module 7, an output module 8, and a controller 9. The conveyor 6 conveys a base 11 between the base storage 2, the resin molding module 3, the plane formation module 4, and the measurement module 5. The conveyor 6 can also convey the base 11 on which a sample 10 (see, for example, FIG. 2) is set, the base 11 that is integrated with the sample 10, the base 11 that is integrated with the sample 10 and processed, and the base 11 that has been measured, in addition to conveying a single body of the base 11.

As illustrated in FIG. 2, the sample 10 is set on the base 11. The base 11 is an example of a setting module, a supporter, or a container of the sample 10. The sample 10 is a component mainly made of synthetic resin material (thermoplastic resin, thermosetting resin, plastics, or engineering plastics). The base 11 is a component mainly made of synthetic resin material (thermoplastic resin, thermosetting resin, plastics, or engineering plastics). The sample 10 and the base 11 can contain organic or inorganic filler. The base 11 illustrated in FIG. 2 is, for example, a component that has a rectangular-solid (cubic) appearance. The sample 10 illustrated in FIG. 2 is a component that has a rectangular (flat rectangular solid) appearance. The sample 10 can be cut into an appropriate shape from a casing (not illustrated) of an electronic equipment or the like, depending on the specification of the base 11.

The base 11 comprises a concave portion (housing or opening) 11b that is opened on one plane 11a. The concave portion 11b comprises a tubular portion 11c and flange portions (second concave portions, supporters, hooks) lid protruding from the tubular portion 11c. The tubular portion 11c is formed in a cylindrical (columnar) shape. The flange portions 11d are formed in keyway (groove) shapes along a direction in which the tubular portion 11c extends. The flange portions 11d are arranged at four positions so as to forma cross shape in a planer view of the plane 11a. Therefore, as illustrated in FIG. 2, the sample 10 can be set on the base 11 such that, for example, end portions of the sample 10 are inserted into the flange portions 11d.

The base storage 2 stores therein a plurality of bases 11. The base storage 2 may store therein the bases 11 having different specifications (size, material, specifications of the concave portion 11b, or the like). The base 11 corresponding to the sample 10 is selected from among the bases 11 stored in the base storage 2 and used in processes in other processing sections (a section, a processor, an area, a processing area, the resin molding module 3, the plane formation module 4, the measurement module 5, or the like).

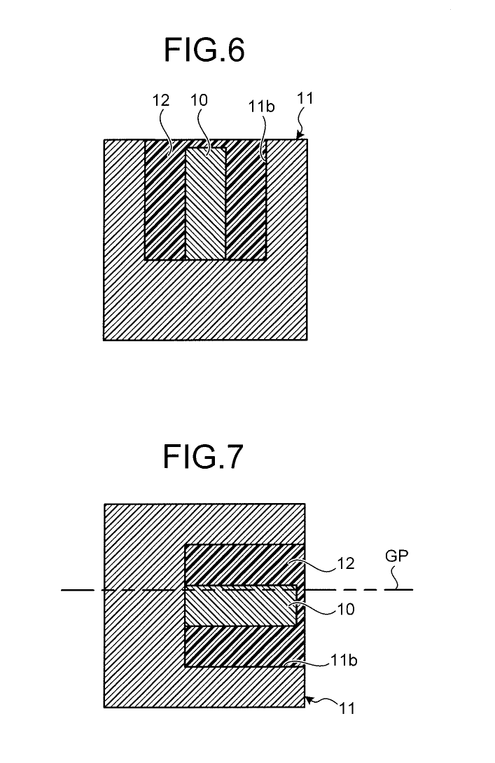

The resin molding module 3 covers at least part of the sample 10 with synthetic resin material (second synthetic resin material, thermoplastic resin, thermosetting resin, plastics, or engineering plastics). In the embodiment, for example, at least part of the sample 10 supported by the base 11 is covered with a cover 12 (see, for example, FIG. 6) made of synthetic resin material.

The plane formation module 4 cuts or grinds the sample 10 to form a plane P1 that is to be an object of measurement (acquisition of physical properties). In the embodiment, for example, the plane P1 is formed on the sample 10 that is supported by the base 11 and that is partly covered with the cover 12.

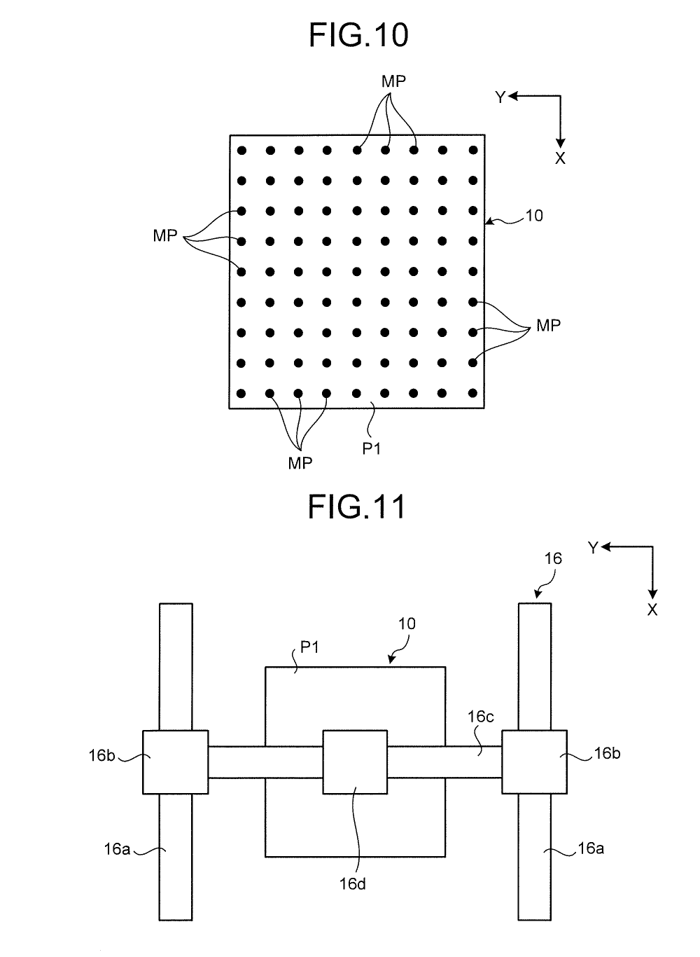

The measurement module 5 presses the plane P1 at a plurality of positions MP (see FIG. 10), thereby acquiring measurement values at the respective positions MP. In the embodiment, for example, the measurement is performed on the plane P1 of the sample 10 that is supported by the base 11, that is partly covered with the cover 12, and that has the plane P1.

The conveyor 6 comprises, as illustrated in FIG. 1, a rail 6a, a slider 6b, an arm 6c, and a supporter 6d. As illustrated in FIG. 1, in the embodiment, for example, the processing sections and the base storage 2 are arranged as a line, and the rail 6a extends along this line. The slider 6b moves back and forth along the longitudinal direction of the rail 6a and stops at least at positions corresponding to (opposing) the processing sections and the base storage 2. The arm 6c is contracted and extended between a contracted state (pulled-in state), in which a tip of the arm 6c is located at a position on the conveying path side (on the rail 6a side) (a first position or a contracted position), and an extended state, in which the tip of the arm 6c is located at a position on the processing sections side or the base storage 2 side (a second position or an extended position). The arm 6c can stop in each of the above states. The supporter 6d switches between a state of supporting the base 11 (holding state) and a state of separating the base 11 (releasing state). Each component of the conveyor 6 has an actuator (not illustrated), such as a motor or a solenoid. The operation of the actuators is controlled by the controller 9. For example, the conveyor 6 controlled by the controller 9 conveys the base 11, i.e., the sample 10, between the processing sections and the base storage 2. The arrangement of the processing sections and the configuration of the conveyor 6 can be changed appropriately. For example, the processing sections may be arranged so as to surround the conveyor 6 or may be stacked in the height direction of the test apparatus 1, or the conveyor 6 may comprise a robot arm or a belt conveyor.

The operation module 7 allows an operator to input operations. The operation module 7 maybe, for example, a keyboard, an operation button, or a switch. The output module 8 performs various types of output. The output module 8 may be, for example, a display, alight emitter, a speaker, or a data writer. The display may be, for example, a liquid crystal display (LED) or an organic electroluminescent display (OELD). The display as the output module 8 outputs measurement values obtained by the measurement module 5 or physical quantities calculated by the controller 9 on the basis of the measurement values, in association with the positions MP. The controller 9 controls each module of the test apparatus 1. The controller 9 comprises, for example, a circuit board and an electronic component mounted on the circuit board. The controller 9 comprises, for example, a central processing unit (CPU) (an example of the controller), a random access memory (RAM) (an example of the memory), a read only memory (ROM) (an example of the memory), a recording medium connector, a recording medium drive, a controller, and a communication interface. The test apparatus 1 operates in accordance with a program (e.g., application). In this case, the CPU operates in accordance with a program that is installed in a nonvolatile storage and that is read from the nonvolatile storage, thereby controlling each module of the test apparatus 1.

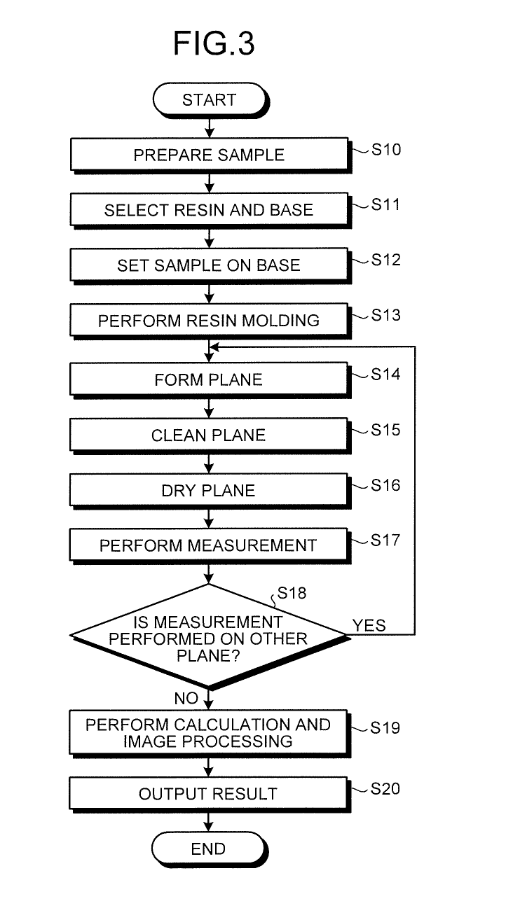

FIG. 3 is a flowchart of an exemplary method for testing the sample 10. First, the sample 10 is prepared (S10). At step S10, for example, the sample 10 is cut out of a component (for example, a casing of an electronic equipment) to be a test object such that the sample 10 matches the base 11. The process at step S10 is performed outside the test apparatus 1. The process at step S10 may be performed in the test apparatus 1 (for example, by the plane formation module 4) when the shape and size of the sample is within a limited range.

The operation module 7 performs input operation to select resin used by the resin molding module 3 (i.e., resin of the cover 12) or to select the base 11 used in the test of the sample 10 (in the process by each section) (S11). The operation module 7 may set parameters or the like in relation to other steps or processes.

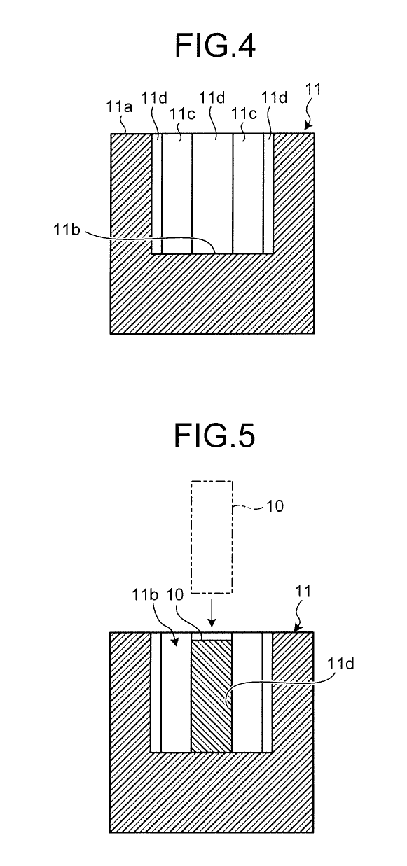

The sample 10 is set on the base 11 (S12). At step S12, for example, as illustrated in FIGS. 4 and 5, an operator inserts the sample 10 into the base 11 conveyed to the resin molding module 3, so that the sample 10 is set on the base 11. In this case, a door (not illustrated) to access the resin molding module 3 from the outside is arranged on the test apparatus 1 in accordance with the resin molding module 3.

The resin molding module 3 covers at least part of the sample 10 with synthetic resin material (second synthetic resin material) (S13). At step S13, for example, as illustrated in FIGS. 5 and 6, fluid synthetic resin material is introduced and filled in the concave portion 11b housing the sample 10, and then the fluid synthetic resin material is solidified, so that at least part of the sample 10 is covered with the cover 12. The cover 12 functions as a supporter for supporting the sample 10. By covering the sample 10 with the cover 12 in this manner, it becomes possible to, for example, easily handle the sample 10 compared with handling a single body of the sample 10. Furthermore, for example, it becomes possible to handle the sample 10 with a smaller size as easy as the sample 10 with a relatively larger size. As the synthetic resin material (second synthetic resin material), material that is solidified at normal temperature with less contraction and less calorific power is preferable. With such material, it becomes possible to prevent the influence of a temperature change or a form change on the property of the sample 10. The resin molding module 3 comprises modules necessary for resin molding (e.g., a resin tank, an injection device, a tube, a nozzle, a temperature change device (temperature adjuster), and the like (not illustrated)).

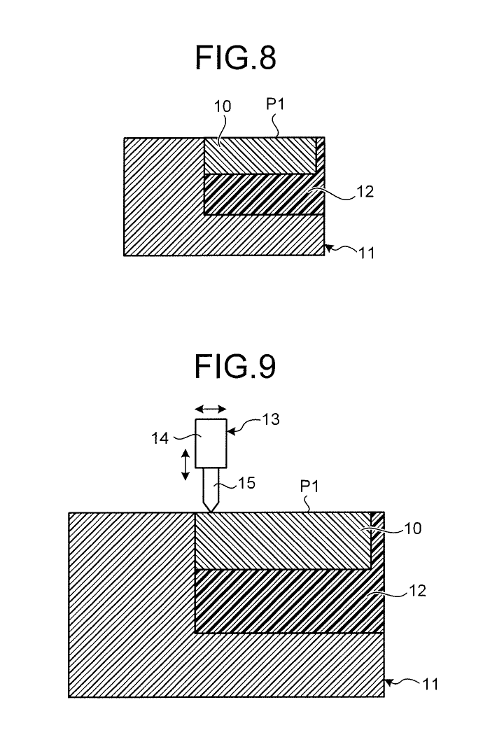

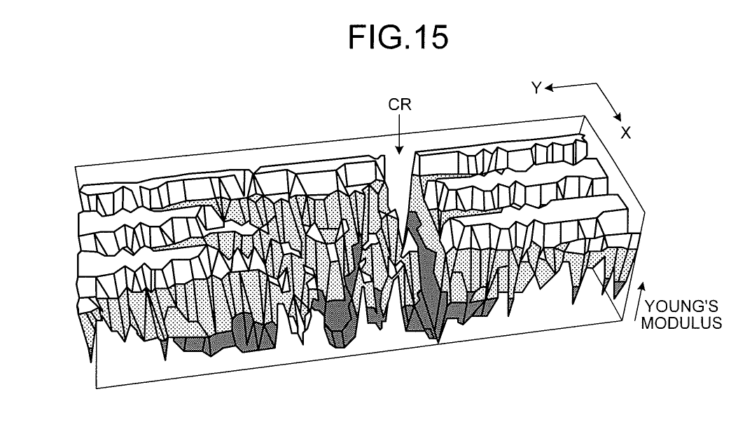

At step S13, it is preferable to cover a side, on which the plane P1 (see FIG. 8) is to be formed at subsequent step S14, with synthetic resin material (second synthetic resin material). Accordingly, for example, it becomes possible to more easily process the plane P1 in the plane formation module 4 even when the surface of the sample 10 is uneven. Furthermore, for example, it becomes possible to fill cracks CR with the synthetic resin material (second synthetic resin material) when the cracks CR occur on the sample 10 (see FIGS. 15 and 16). Therefore, for example, it becomes possible to prevent any inconvenience that may occur due to the cracks CR when the test apparatus 1 handles the sample 10 (e.g., extension of the cracks CR or cracking of the sample 10). When there are narrow gaps or an infinite number of thin cracks CR on the sample 10, it may be possible to perform vacuum defoaming.

The synthetic resin material (second synthetic resin material) that covers the sample 10 at step S13 may be different from the material of the sample 10. Specifically, the synthetic resin material of the sample 10 and the synthetic resin material of the cover 12 may be different from each other in terms of, for example, physical properties, characteristics, hardness, degree of hardness, Young's modulus, synthetic resin substrates, presence or absence of filler, content rate of filler, or types of filler. When cracks CR or the like occur on the plane P1 or the like of the sample 10, and if fluid synthetic resin material (second synthetic resin material) is introduced at step S13, the synthetic resin material is introduced into the cracks CR as described above. In this case, if the synthetic resin material introduced into the cracks CR is different from the synthetic resin material of the sample 10, it becomes easy to distinguish between a measurement result of a portion other than the cracks CR and a measurement result of the cracks CR portions (portions into which the synthetic resin material is introduced), which are obtained by the measurement module 5. Therefore, for example, it becomes possible to prevent error in detection of abnormal portions, enabling to easily perform the test (measurement) with higher accuracy.

When the synthetic resin material covering the sample 10 (i.e., the second synthetic resin material making up the cover 12) is harder than the material of the sample 10 (with higher hardness and larger Young's modulus), the hardness of the test apparatus 1 (assembly), in which the sample 10 and the cover 12 are integrated with each other, increases. Therefore, for example, the sample 10 can be more firmly supported by the base 11 or the cover 12, so that it becomes possible to easily reduce the influence of the cover 12 on the measurement result obtained by the measurement module 5. Furthermore, for example, it becomes possible to prevent occurrence of any inconvenient situation in which, for example, the cover 12 is partly flaked off because the cover 12 is too soft, during processes performed by the plane formation module 4.

On the other hand, when the synthetic resin material covering the sample 10 (i.e., the second synthetic resin material making up the cover 12) is softer than the sample 10 (with lower hardness and smaller Young's modulus), the crack CR portions become soft. Therefore, for example, the hardness decreases at the portions where the cracks CR occur, compared with portions where no crack CR occurs. As a result, it becomes possible to easily distinguish between the measurement results, compared with a case that the hard synthetic resin material is introduced into the cracks CR and the hardness of the portions with no crack CR is increased.

The plane formation module 4 cuts or grinds the sample 10 to form the plane P1 (S14). At step S14, for example, as illustrated in FIGS. 7 and 8, the base 11 (assembly) integrated with the sample 10 and the cover 12 is cut along a plane GP that is along a plane at which the sample 10 is to be tested, and then the plane GP is grinded, so that the plane P1 is formed. With the process at step S14, it becomes possible to easily prevent the influence of the irregularities of the plane P1 (which may cause error in the measurement) on the measurement results. It may be possible to omit the cutting depending on the specifications of the sample 10 or the base 11 (assembly). In the grinding, a rotary grinder or the like may be used as a grinding module. In the grinding, the grinding module (e.g., grinder) may be changed so that the surface roughness can be gradually reduced. For example, the plane P1 is mirror-finished such that the irregularities are reduced to 500 nanometers (nm) or smaller (or preferable, in a range of 10 to 20 nm) in the end of the process.

The plane P1 is cleaned (S15). When the plane P1 gets wet at S15, the plane P1 is dried (S16). At Steps S15 and S16, a cleaner (cleaning module (not illustrated)) and a drier (drying module (not illustrated)) are arranged inside the test apparatus 1 (e.g., in at least one of the resin molding module 3 and the plane formation module 4). For example, at least P1 of the base 11 (assembly) integrated with the sample 10 and the cover 12, or preferably, the whole assembly, is cleansed and dried. The cleaner is configured as, for example, a device that supplies water with low impurity content (pure water), and comprises a tank, a tube, a nozzle, a pump, and the like. The drier comprises, for example, a case, a heater, a fan, and a nozzle. When the cleaner and the drier are arranged in the resin molding module 3, it is advantageous in that, for example, at least part of a temperature change mechanism used for resin molding can also be used as the drier. When the cleaner and the drier are arranged in the plane formation module 4, it is advantageous in that, for example, adhesion of refuse or the like is less likely to occur compared with a configuration in which the cleaner and the drier are arranged in a different processing section and the base is conveyed from this processing section to the plane formation module 4.

The measurement module 5 presses the plane P1 at the positions MP and obtains measurement values at the respective positions MP (S17). At step S17, as illustrated in FIG. 9, a measurement device 13 comprises, for example, a base module (a supporter or a casing) 14 and a movable module (a presser or a pressurizer) 15. The base module 14 supports the movable module 15 so that the movable module 15 can move back and forth along an approaching direction toward the plane P1 (or a backward direction, i.e., a direction crossing (perpendicular to) the plane P1). The base module 14 applies load to the movable module 15 so that the movable module 15 is pressed against the plane P1. That is, the base module 14 comprises an actuator (e.g., an electromagnetic solenoid (not illustrated)). The base module 14 also comprises a displacement sensor that detects the amount of displacement of the movable module 15 (e.g., an capacitance displacement meter (not illustrated)). The movable module 15 is an example of the presser.

As illustrated in FIG. 10, at step S17, for example, the positions MP at which the measurement is performed are discretely arranged in a matrix form (a matrix, or a grid, in the two-dimensional coordinate with the X direction and the Y direction that are perpendicular to each other) on the plane P1 of the sample 10. To perform the measurement at the positions MP described above, the measurement module 5 comprises, for example, a moving device 16 that moves the measurement device 13 as illustrated in FIG. 11. The moving device 16 comprises, for example, rails 16a, sliders 16b, a rail 16c, and a slider 16d. As illustrated in FIG. 11, in the embodiment, the moving device 16 is located so as to cover the plane P1 of the sample 10 (e.g., above the plane P1). The rails 16a and the rail 16c extend along crossing directions (directions perpendicular to each other) along the plane P1. In the example in FIG. 11, the rails 16a extend in the X direction and the rail 16c extends in the Y direction that is perpendicular to the X direction. The sliders 16b are movable back and forth along the longitudinal direction of the respective rails 16a and are stoppable at each of positions (arbitrary positions). The slider 16d is movable back and forth along the longitudinal direction of the rail 16c and is stoppable at each of positions (arbitrary position). The measurement device 13 as illustrated in FIG. 9 is mounted on the slider 16d. Each module of the moving device 16 comprises an actuator (not illustrated), such as a motor or a solenoid. The operations of the actuators are controlled by the controller 9. With this configuration, the moving device 16 can move the measurement device 13 to the positions corresponding to (opposing to) the positions MP. The arrangement of the positions MP and the configuration of the moving device 16 can be changed appropriately. It is possible to configure the sample 10 and the base 11 so that they can move relative to the fixedly-arranged measurement device 13. Alternatively, it is possible to configure the measurement device 13 and the assembly of the sample 10 and the base 11 so that both of them can move relative to each other. In this case, the moving device (not illustrated) arranged on the measurement module 5 may move the sample 10 and the base 11, or the conveyor 6 may move the sample 10 and the base 11. The moving device 16 of the measurement device 13, the moving device (not illustrated) of the sample 10 and the base 11, or the conveyor 6 can move in a direction perpendicular to the X direction or the Y direction (a direction in which the assembly of the sample 10 and the base 11 comes close to or away from the measurement device 13, or the direction normal to the plane P1), instead of moving in the X direction or the Y direction.

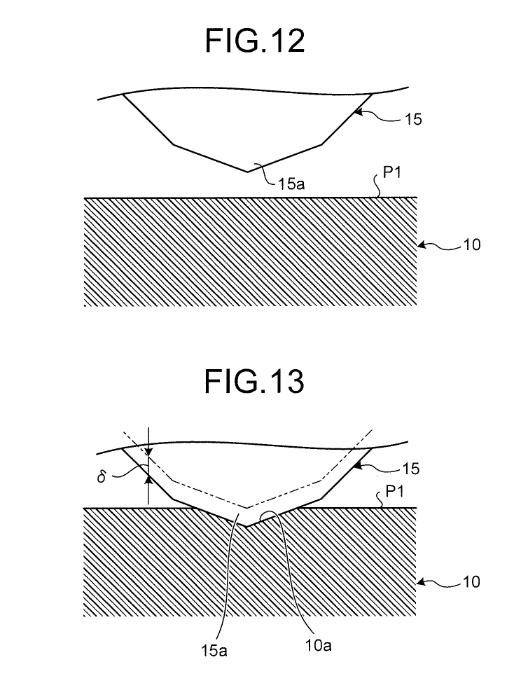

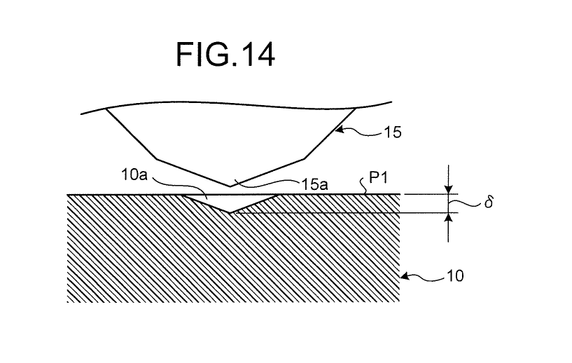

At step S17, a minute portion is measured by, for example, nanoindenter (nanoindentation). As illustrated in FIGS. 12 to 14, at step S17, the measurement device 13 causes the movable module 15 to come close to, come into contact with, and be pushed into the plane P1. For example, the movable module 15 moves in a direction perpendicular to the plane P1. The positions MP on the plane P1 are dented by being pressed by a tip 15a of the movable module 15. The measurement device 13 applies known load (predetermined load or force, e.g., load of the same magnitude is applied to each of the positions MP). Therefore, the insertion of the movable module 15 into the sample 10 stops at the deepest position according to the physical properties (e.g., hardness or elasticity) of the sample 10. That is, a concave portion 10a is formed on the plane P1 because the movable module 15 presses the plane P1 by the tip 15a. The depth of the concave portion 10a varies depending on the physical properties (e.g., hardness or elasticity). The concave portion 10a becomes shallower as the hardness of the position MP becomes harder (as the hardness or the Young's modulus increases). The magnitude of the load is appropriately set so that the depth of the concave portion 10a becomes a few nm or deeper (at least 1 nm). The displacement sensor detects a displacement amount of the movable module 15 when the plane P1 is dented. For example, the displacement sensor detects the displacement amount δ from a position where the tip 15a of the movable module 15 is in contact with the plane P1 to a position where the tip 15a reaches the deepest position. The displacement amount δ is the same as a depth δ of the concave portion 10a. For example, the measurement value obtained at step S17 is the displacement amount δ (the depth of the concave portion 10a). The tip 15a is formed in, for example, a multi-sided pyramid shape (e.g., triangular pyramid shape) or a cone shape. At least the tip 15a of the movable module 15 is made of hard material, such as diamond.

When the measurement is not performed on a plane other than the plane P1 (No at S18), a predetermined physical quantity at each of the positions MP is calculated on the basis of the measurement values obtained at step S17 (S19). At step S19, for example, the controller 9 calculates a physical quantity (e.g., hardness or Young's modulus) corresponding to the physical properties (e.g., hardness or elasticity) at each of the positions MP, on the basis of the displacement amount δ obtained at step S17, the magnitude of the load applied by the movable module 15, and the shape of the tip 15a of the movable module 15. For example, when the tip 15a has the shape as illustrated in FIGS. 12 to 14, a contact area between the tip 15a and the sample 10 with the plane P1 dented can be calculated geometrically from the displacement amount δ. Therefore, the controller 9 can calculate the physical quantity (e.g., hardness or Young's modulus) corresponding to the physical properties (e.g., hardness or elasticity) at each of the positions MP, on the basis of the calculated contact area and the given magnitude of the load.

At step S19, image processing corresponding to output forms is also performed. Then, an output result, in which the measurement values or the physical quantities calculated based on the measurement values are associated with the respective positions MP, is output (S20). At step S20, for example, a graph as illustrated in FIG. 15 is displayed on a display screen of a display serving as the output module 8. FIG. 15 is an example of the output result (graph) that represents Young's modulus as the physical quantities at the respective positions MP that are discretely arranged in a two dimensional array with the X direction and the Y direction. In the example in FIG. 15, spatial interpolation is performed and contour lines of Young's modulus are illustrated. Young's modulus is high at white-colored portions and Young's modulus becomes lower as the density of dots in dot patterns increases. FIG. 16 is a perspective view schematically illustrating the sample 10 corresponding to the output example in FIG. 15. When comparing FIG. 15 and FIG. 16, it becomes easy to visually recognize portions (regions) where weld lines WL and cracks CR occur on the two-dimensional XY coordinate of the plane P1 of the sample 10. FIG. 15 is a mere example of the output result obtained at step S20; therefore, the form of the output result is not limited to this example. For example, the output result may be output as a plan view in which results are distinguished by colors or patterns. Furthermore, in the example in FIG. 15, it is observed that the weld lines WL are present at locations where Young's modulus becomes lower than a normal portion; however, the weld lines may be observed at locations where Young s modulus is higher than the normal portion. That is, according to the test method and the test apparatus of the embodiment, a portion (region) where abnormality occurs in the sample 10 can be recognized as a portion (region) having a different physical quantity from that of a normal portion.

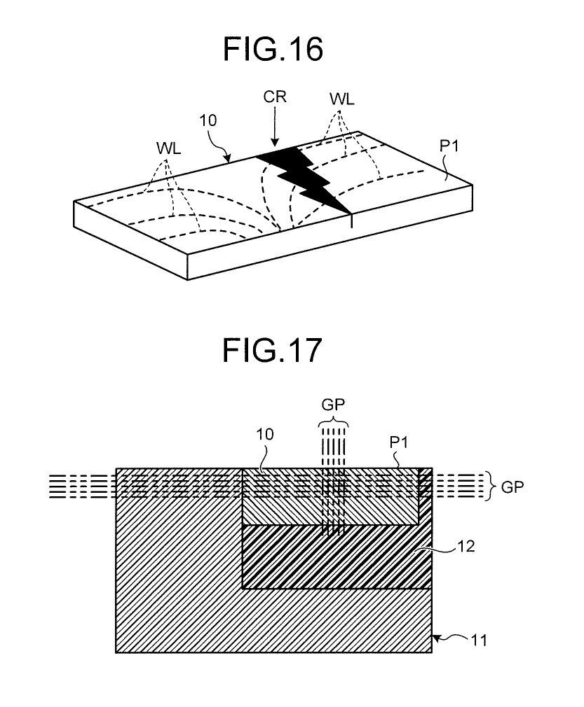

When the measurement is performed on a plane other than the plane P1 (Yes at S18), the process returns to step S14, at which another plane is formed. Thereafter, the processes from step S15 are repeated on the formed plane. As illustrated in FIG. 17, the base 11 (assembly) integrated with the sample 10 and the cover 12 is cut along the plane GP that is along a plane at which the sample 10 is to be tested, and then the plane GP is grinded, so that another plane is formed. That is, the sample 10 is cut or grinded at the plane GP, so that a plane to be a measurement object is formed. Then the measurement is performed on the plane, and after the measurement, a new plane GP is further cut or grinded, so that a new plane as a measurement object is formed. Thereafter, the measurement is performed on the new plane. That is, in the embodiment, the sample 10 is gradually cut and grinded to form planes, and a measurement value and a physical quantity of each plane are obtained, so that the physical quantity (e.g., hardness or Young's modulus) corresponding to the physical properties (e.g., hardness or elasticity) of even the interior of the sample 10 can be tested.

As described above, the synthetic resin material test method of the embodiment comprises: forming the plane P1 by cutting or grinding the sample 10 made of synthetic resin material; obtaining a measurement value at each of the positions MP on the plane P1 by pressing the plane P1 at each of the positions MP; and outputting the measurement value or a physical quantity calculated based on the measurement values, in association with each of the positions MP. Therefore, for example, it is possible to obtain the measurement values at the respective positions MP on the plane P1 with higher accuracy. Furthermore, it is possible to easily recognize the physical quantity at each of the positions MP on the plane P1. Consequently, it is possible to more easily recognize a region where abnormality occurs in the sample 10 made of synthetic resin material, with higher accuracy.

Furthermore, in the embodiment, at the obtaining the measurement value, the displacement amount of the movable module 15 from the plane P1 is measured when the movable module 15 is pressed against the plane P1, and a physical quantity corresponding to the hardness or the elasticity of the sample 10 is calculated on the basis of the load by which the movable module 15 is pressed against the plane P1. Therefore, for example, it is possible to obtain the physical quantity corresponding to the local hardness or the local elasticity of the sample 10, i.e., the physical quantity corresponding to the hardness or the elasticity of the sample 10, with higher accuracy.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions. For example, it is possible to calculate the physical quantity other than the hardness or Young's modulus from the measurement values, and obtain a distribution of the physical quantity of the sample. Furthermore, the specifications of the test apparatus and modules of the test apparatus, i.e., a base storage, a resin molding module, a plane formation module, a measurement module, a conveyor, an operation module, an output module, a controller, a sample, a base, a cover, a measurement device, a base module, a movable module, and a moving device (structure, orientation, shape, size, length, width, thickness, height, number, arrangement, position, material, and the like) can be appropriately changed in embodiments. Furthermore, the output module may be a data output device that outputs data to a storage medium or may be a printer that prints the output results on media, such as papers. In the embodiment, a case is explained that the sample, the base, and the cover are made of synthetic resin material. However, the embodiments can be similarly applied to any material (e.g., metal material) other than synthetic resin material.

Moreover, the various modules of the systems described herein can be implemented as software applications, hardware and/or software modules, or components on one or more computers, such as servers. While the various modules are illustrated separately, they may share some or all of the same underlying logic or code.

What is claimed is:

1. A synthetic resin material test method comprising:
forming a first plane on a sample made of first synthetic resin material by cutting or grinding the sample;
obtaining measurement values at a plurality of positions on the first plane by pressing the first plane at the positions; and
outputting the measurement values or physical quantities calculated based on the measurement values, in association with the respective positions.

2. The synthetic resin material test method of claim 1, wherein
the obtaining comprises measuring, as the measurement values, a displacement amount of a presser from the first plane when the presser is pressed against each of the positions on the first plane, and
the physical quantities are calculated in accordance with hardness or elasticity of the sample, on the basis of a shape of the presser, the displacement amount, and load caused by the presser when the presser presses the first plane.

3. The synthetic resin material test method of claim 1, further comprising covering at least a portion of the sample with second synthetic resin material, before the first plane is formed at the forming.

4. The synthetic resin material test method of claim 3, wherein the forming comprises forming the first plane on the portion covered with the second synthetic resin material.

5. The synthetic resin material test method of claim 3, wherein hardness of the second synthetic resin material is different from hardness of the first synthetic resin material.

6. The synthetic resin material test method of claim 5, wherein the second synthetic resin material is softer than the first synthetic resin material.

7. The synthetic resin material test method of claim 5, wherein the second synthetic resin material is harder than the first synthetic resin material.

8. The synthetic resin material test method of claim 3, wherein
the covering the sample with the second synthetic resin material comprises:
introducing the second synthetic resin material in a fluid state into a container housing the sample; and
solidifying the introduced second synthetic resin material.

9. The synthetic resin material test method of claim 1, further comprising:
forming a second plane by cutting or grinding the first plane after the measurement values of the first plane are measured at the obtaining; and
obtaining measurement values at a plurality of positions on the second plane by pressing the second plane at the positions.

10. A material test method comprising:
forming a first plane on a sample by cutting or grinding the sample;
obtaining measurement values at a plurality of positions on the plane by pressing the first plane at the positions; and
outputting the measurement values or physical quantities calculated based on the measurement values, in association with the respective positions.

11. A synthetic resin material test apparatus comprising:
a plane formation module configured to form a first plane on a sample made of first synthetic resin material by cutting or grinding the sample;
a measurement module configured to obtain measurement values at a plurality of positions on the first plane by pressing the first plane at the positions; and
an output module configured to output the measurement values or physical quantities calculated based on the measurement values, in association with the respective positions.
